(19) United States
(12) Patent Application Publication
(10) Pub. No.: US 2011/0064833 A1
(43) Pub. Date: Mar. 17, 2011

(54) SYNERGISTIC HERBAL COMPOSITION AND PROCESS OF PREPARATION THEREOF

(76) Inventors: Villoo Morawala Patell, Bangalore (IN); Jagadeesh Badamaranahalli Henjarappa, Bangalore (IN); Chandramohan Bangalore Siddaiah, Bangalore (IN); Rajesh Ullant, Bangalore (IN)

(21) Appl. No.: 12/740,682

(22) PCT Filed: Dec. 11, 2007
(86) PCT No.: PCT/IN2007/000581

§ 371 Date: Apr. 29, 2010

(30) Foreign Application Priority Data
Oct. 29, 2007 (IN) 2449/CHE/2007

(51) Int. Cl. A61K 36/61; A61P 3/10

(52) U.S. Cl. 424/739

(57) ABSTRACT

The present invention relates to a synergistic herbal composition for diabetes and related disorders comprising extracts of plant Eugenia of concentration ranging between 10-80% and plant Cinnamomum of concentration ranging between 10-80% optionally along with pharmaceutically acceptable excipients; a method of treating diabetes and related disorders in a subject in need thereof by administering the composition comprising extracts of plant Eugenia of concentration ranging between 10-80% and plant Cinnamomum of concentration ranging between 10-80% optionally along with pharmaceutically acceptable excipients; and a process for preparation of a synergistic herbal composition for diabetes and related disorders, wherein the process comprising steps of: (a) obtaining extracts of Eugenia and Cinnamomum separately using aqueous or organic solvents; and (b) formulating the extracts to arrive at the synergistic composition.

### FIELD OF INVENTION

The present invention relates to a herbal composition with antidiabetic effect. The invention also relates to clinically safe and non-toxic plant extracts useful for the treatment of diabetes and related disorders. The invention further relates to a synergistic mixture of plant extracts for the treatment of diabetic and related disorders. In particular, the invention relates to an easily administrable mixture of extracts obtained from Eugenia sp and cinnamon sp. The invention thereafter discloses easily administrable mixture of plant extract, which can be safely administered both to children and adult.

The present invention also relates to a process for the preparation of the mixture of plants extract wherein each of the particular plant extract is added in an appropriate ratio.

### BACKGROUND AND PRIOR ART OF THE INVENTION

Diabetes is a metabolic disorder characterized by hyperglycemia (high glucose blood sugar). It is a disease wherein the body does not produce or improperly uses insulin. The cause of diabetes continues to be a mystery but lack of excursion and obesity appears to play a major role in diabetes. Diabetes is characterized by hyperglycemia, glycosuria, hyperlipidemia, negative nitrogen balance and sometimes ketonemia. According to world health organization there are three types of diabetes.

Type 1—Type 1 diabetes or insulin-dependent diabetes mellitus (IDDM) is the form in which destruction of beta cells results in the production of impaired insulin. The type 1 diabetes is usually diagnosed in children, young adults and it was previously known as juvenile diabetes. This type of diabetes is most commonly classified as an autoimmune disorder resulting from an infection in people whose immune system is genetically predisposed to develop a vigorous autoimmune response against pancreatic beta cell antigens.

Type 2—The type 2 diabetes is most common form of diabetes. In this type, either the body does not produce enough insulin or the cells ignore insulin. It is well known that insulin is necessary for the body for using glucose. Glucose is the basic fuel for body cells. An increase in blood glucose level causes problems. These problems include starvation of body cells. In other cases a high blood glucose level may hurt eyes, kidneys, nerves or heart.

Gestational diabetes—This type of diabetes generally occurs in pregnant women. These patients have never had diabetes earlier in their lives. The disease starts when body is unable to make and use all the insulin it needs during pregnancy. Without enough insulin, glucose cannot leave the blood and be changed to energy. Glucose builds up in the blood to high levels and thus hyperglycemia is caused.

Type 2 diabetes increases risk of many serious complications. Some of the complications related to type 2 diabetes are cardiovascular disease, blindness, neuropathy and nephropathy. People with type 2 diabetes require oral anti diabetic agents for satisfactory blood glucose control. At least one third must however take insulin for reducing blood glucose levels.

Various treatments are available for diabetes. These include parental administration of insulin, antidiabetic drugs to lower blood sugar level, herbal and ayurvedic extracts. Insulin cannot be administrated orally. It being a protein, the acids in the stomach destroy it when orally administered. Even if it makes through the stomach, the digestive enzyme secreted by the digestive part of the pancreas digest it. Therefore, administration of insulin orally in not suitable.

Various anti diabetic drugs are available for the treatment of diabetes. These include the categories of sulfonylureas, meglinides, bigunides, alpha-glucosidase inhibitors, Thiazilinediones, D-phenylalanine derivatives. These drugs stimulate the insulin production. These drugs however do not come without the side effects associated with them. Insulin production by the drugs may not happen at the right time or at other times it may be excessive causing an undesirable fall in blood glucose level or hypoglycemia. A long-term use of these anti diabetic drugs may cause degeneration of insulin granules.

Ayurveda offers various treatments for diabetes (Madhumeha). In ayurveda diabetes is described as a metabolic kapha type of disorder in which diminished function of agni leads to a tendency towards high blood sugar. The main cause of the disease is fat, urine and kapha build ups due to foods, liquids, lifestyle and others. Herbal ingredients such as Bitter Melon (Momordica Charantia), Gurmar (Gymnema Sylvester), onion and garlic (Allium cepa and Allium sativum), Fenugreek (Trigonella Foenum), Blueberry leaves (Vaccinium Myrtillus), Asian Ginseng, Stevia etc are few examples in this category.

There are various advantage of using herbal medicine or herbal composition. Herbal compositions are naturally available. They have with minimal or no side effects. They have lower cost of production and offer greater efficacy than existing therapies and medications.

Eugenia sp are used as household remedy for diabetes. Commonly Eugenia is known as java plum, black plum, jambul and blackberry. It grows naturally in clayey loam soil in tropical as well as sub-tropical zones. These plants are widely cultivated in Haryana as well as rest of the indo-gangetic plains on large scale. Its habitat starts from Myanmar and extends up to Afghanistan. Eugenia also forms constituents of many herbal formulations for diabetes marketed in India. It is known that administration of the ethanolic extract of Eugenia at a concentration of 100 mg/kg of body weight significantly decreases the level of blood glucose.

Cinnamomum is another well known herb which has historical medicinal use in different diseases. Cinnamomum is one of the oldest herbal medicine known, having been mentioned in Chinese texts as long as 4,000 years ago. Cinnamon sp. has been found to have a positive effect on glycemic control and the lipid profile in patients with diabetes. Cinnamomum contains biological components that are believed to have potential for treatment of diabetes. The main properties of cinnamomum are astrigent, warming, stimulant, carminative, antiseptic, antifungal, anti-viral, blood purifier and digestive aid. All these properties make it a good medicinal plant.

The background information is provided for the purpose of making known information believed by the applicant to be of possible relevance to the present invention. No admission is necessarily intended, nor should be construed, that any of the preceding information constitutes prior art against invention.

### OBJECTS OF THE INVENTION

The main object of the present invention is to obtain a synergistic herbal composition for diabetes and related disorders.

Another main object of the present invention is to obtain a synergistic herbal composition comprising extracts of plant Eugenia of concentration ranging between 10-80% and plant Cinnamomum of concentration ranging between 10-80%.

Yet another object of the present invention is to obtain a method of treating diabetes and related disorders in a subject in need thereof by administering the composition.

Still another object of the present invention is to obtain a process for preparation of a synergistic herbal composition for diabetes and related disorders.

### STATEMENT OF THE INVENTION

The present invention relates to a synergistic herbal composition for diabetes and related disorders comprising extracts of plant Eugenia of concentration ranging between 10-80% and plant Cinnamomum of concentration ranging between 10-80% optionally along with pharmaceutically acceptable excipients; a method of treating diabetes and related disorders in a subject in need thereof by administering the composition comprising extracts of plant Eugenia of concentration ranging between 10-80% and plant Cinnamomum of concentration ranging between 10-80% optionally along with pharmaceutically acceptable excipients; and a process for preparation of a synergistic herbal composition for diabetes and related disorders, wherein the process comprising steps of: (a) obtaining extracts of Eugenia and Cinnamomum separately using aqueous or organic solvents; and (b) formulating the extracts to arrive at the synergistic composition.

### BRIEF DESCRIPTION OF ACCOMPANYING FIGURES

The present invention can be illustrated more conveniently by detailed description given below and the full drawing which are listed below.

FIG. 1: Bar graph showing free radical scavenging ability of an exemplary plant mixture named SM-9.

FIG. 2: Total polyphenol estimation of an exemplary plant mixture named SM-9

FIG. 3: Bar graph showing change in the post-prandial glucose levels in rats with pioglitazone and SM-9 intervention.

FIG. 4: Bar graph showing changes in HbA1C levels of rats with pioglitazone, glibenclamide and SM-9 intervention.

FIG. 5: Bar graph showing percentage insulin mimetic activities shown by the target plant extract mixture as determined in adipocytes.

FIG. 6: Bar graph showing percentage insulin sensitization activities shown by the plant extract mixtures as compared to individual plant extracts determined in adipocytes.

FIG. 7: Bar graph showing percentage insulin mimetic activity of different plant extract shown in myocytes.

FIG. 8: Bar graph showing percentage insulin sensitization activity of different plant extract shown in myocytes.

### DETAILED DESCRIPTION OF THE INVENTION

The present invention relates to a synergistic herbal composition for diabetes and related disorders comprising extracts of plant Eugenia of concentration ranging between 10-80% and plant Cinnamomum of concentration ranging between 10-80% optionally along with pharmaceutically acceptable excipients.

In another embodiment of the present invention, the extract is obtained from plant parts selected from a group comprising root, shoot, leaf, seed, and fruit or the whole plant.

In yet another embodiment of the present invention, the extract is obtained from seeds of the plants.

In still another embodiment of the present invention, the extract is either an aqueous or an organic extract.

In still another embodiment of the present invention, the extract is an aqueous extract.

In still another embodiment of the present invention, the extract is an alcoholic extract.

In still another embodiment of the present invention, the extract is an ethanolic extract.

In still another embodiment of the present invention, the excipients are selected from a group comprising additives, gums, sweeteners, coatings, binders, disintegrants, lubricants, disintegration agents, suspending agents, granulating agents, solvents, colorants, glidants, anti-adherents, anti-static agents, surfactants, plasticizers, emulsifying agents, flavoring agents, viscocity enhancers and antioxidants.

In still another embodiment of the present invention, the composition is formulated into dosage forms selected from a group comprising liquid, troches, lozenges, powder, granule, capsule, tablet, patch, gel, emulsion, cream, lotion, dentrifice, spray, drop, suspension, syrups, elixirs, phyotceuticals and neutraceuticals.

In still another embodiment of the present invention, the composition is free of side effects.

The present invention also relates to a method of treating diabetes and related disorders in a subject in need thereof by administering the composition comprising extracts of plant Eugenia of concentration ranging between 10-80% and plant Cinnamomum of concentration ranging between 10-80% optionally along with pharmaceutically acceptable excipients.

In still another embodiment of the present invention, the related disorders comprise diabetic acidosis, diabetic xanthoma, diabetic myatrophy, diabetic ketosis, diabetic coma, diabetic stomach disorders, diabetic gangrene, diabetic ulcer, diabetic diarrhea, diabetic microangiopathy, diabetic uterosclerosis, diabetic cardiomyopathy, diabetic neuropathy, diabetic nephropathy, diabetic blister, diabetic cataract, diabetic dermatitis, diabetic scleredema, diabetic retinopathy, diabetic necrobiosis lipoidica, diabetic and blood flow obstructions.

In still another embodiment of the present invention, the subject is an animal including human beings.

In still another embodiment of the present invention, the excipients are selected from a group comprising additives, gums, sweeteners, coatings, binders, disintegrants, lubricants, disintegration agents, suspending agents, granulating agents, solvents, colorants, glidants, anti-adherents, anti-static agents, surfactants, plasticizers, emulsifying agents, flavoring agents, viscocity enhancers and antioxidants.

In still another embodiment of the present invention, the composition is formulated into dosage forms selected from a group comprising liquid, troches, lozenges, powder, granule, capsule, tablet, patch, gel, emulsion, cream, lotion, dentrifice, drop, suspension, syrups, elixirs, phyotceuticals and neutraceuticals.

In still another embodiment of the present invention, the composition is free of side effects.

The present invention also relates to a process for preparation of a synergistic herbal composition for diabetes and related disorders, wherein the process comprising steps of:
a) obtaining extracts of Eugenia and Cinnamomum separately using aqueous or organic solvents; and
b) formulating the extracts to arrive at the synergistic composition.

In still another embodiment of the present invention, the extract is obtained from plant parts selected from a group comprising root, shoot, leaf, seed, and fruit or the whole plant.

In still another embodiment of the present invention, the extract is obtained preferably from seeds of the plants.

In still another embodiment of the present invention, the organic solvents are selected from a group comprising heterocyclic aromatic compounds, aliphatic compounds, ketones, cyanides, alcohols, nitriles, esters, ether and mixtures of one or more thereof.

In still another embodiment of the present invention, the organic solvent is ethanol.

In still another embodiment of the present invention, the extract obtained are treated with organoleptic agents selected from a group comprising decolorizing agents, deodorizing agents and debitterizing agents.

In still another embodiment of the present invention, the decolorizing agents are selected from a group comprising peroxides, carbon, metals and organic solvents.

In still another embodiment of the present invention, the decolorizing agent is preferably hydrogen peroxide.

In still another embodiment of the present invention, the deodorizing agents are selected from a group comprising peroxides, carbon and organic solvents.

In still another embodiment of the present invention, the deodorizing agent is preferably ketone.

In still another embodiment of the present invention, the composition is formulated into dosage forms like liquid, troches, lozenges, powder, granule, capsule, tablet, patch, gel, emulsion, cream, lotion, dentrifice, drop, suspension, syrups, elixirs, phyotceuticals and neutraceuticals.

The present invention provides a novel anti-diabetic mixture obtained from plant extracts. Further the invention provides a novel herbal composition comprising an effective amount of mixture of plant extracts obtained from the plant species. The present invention is suited for treatment and prevention of diabetes as commonly experienced in mammals, and particularly humans.

As used herein, the term “potential plant” includes from which antidiabetic extract can be extracted out. The term comprises of plant like Momorice sp, Salacia sp, Eugenia sp, Coccimia, Cinammomum sp, Gynnema sp, Pterocapus sp, Azadiricta sp and Trigonella sp.

As used herein, “anti-diabetic” or “hypoglycemic” compound or composition generally refers to an agent that lowers blood glucose levels. If blood glucose level is decreased by at least about 100 mg/dl, then the compound is considered to be a hypoglycemic agent. The hypoglycemic or anti-diabetic effect can be measured by a variety of methods including, but not limited to, measuring the blood glucose levels, the rate of insulin binding to its receptor, the level of insulin secretion from pancreatic beta cells, and inhibition of glucohydrolase activity.

As used herein the term, “related disorders” means disorders related to diabetes mellitus, which include but are not limited to Diabetic Neuropathy, Diabetic Diarrhoea, Urinary retention, Gustatory Swelling, Papillary Reflexes, Cardiac Autonomic Disturbances, Collagen Disturbances, thickening of capillary basement membrane, increase in vessel wall matrix and cellular proliferation resulting in vascular complications like lumen narrowing, early atherosclerosis, sclerosis of glomerular capillaries, retinopathy, neuropathy and peripheral vascular insufficiency.

As used herein the “treated extract” refers to the dried extract treated with deodourizing, decolorizing agents which include but are not restricted to hydrogen peroxide, ketone, carbon, metals etc.

As used herein the “composition” means any administrable from of the herbal extract, ayurvedic or pharmaceutical composition. The term includes but is not restricted to tablets, capsules, lozenges, creams, and lotions.

In accordance with an embodiment of the present invention, the potential plant is a member of the family Myrtaceae. Other mixtures of plant extracts capable of showing positive anti-diabetic activity could be isolated using similar techniques from other potential plants. The present invention is directed to a herbal medicinal composition comprising the foregoing potential plant extracts that can be administered to a person suffering from diabetes.

In accordance with another embodiment of the present invention, the potential plant is a member of the family Lauraceae. Other mixtures of plant extracts capable of positive anti-diabetic properties could be isolated using similar techniques from other potential plants. The present invention is directed to a herbal medicinal composition comprising the foregoing potential plant extracts that can be administered to a person suffering from diabetes.

The crux of the invention lies in the development of plant extract mixtures isolated from various potential plant species and in particular Cinnamomum sp and Eugenia sp. In further embodiment, the invention states a mixture composition containing an optimum concentration of Eugenia and Cinnamomum plant extract. These extracts have been herein added in various ratios. The ratios are the result of planned experiment conducted to optimize the hypoglycemic potentials resident in the plant extracts.

A particular embodiment of the present invention further relates to a method of preparation of plant extracts for treating and preventing diabetes and its associated conditions. It is noteworthy that various plant extracts obtained by the extraction procedure laid down hereunder show unprecedented insulin sensitization activities and insulin mimetic activities in various cells and tissues. Additionally, the alpha glucosidase inhibitory activity, shown by these extracts is invariably enhanced and is evident in all plant extracts.

The plant extracts however differ in the degree of anti-diabetic activity shown. For example, Eugenia extracts show the best of alpha glucosidase inhibitory activity amongst the extracts. The insulin sensitization activity is best shown amongst all by Cinnamomum extract. Also, it is seen that the extraction solvent used makes a difference in the activities shown. Therefore a need remains for a desired synergistic plant extract mixture, which shows for example the alpha glucosidase activity of Eugenia extract and the insulin sensitization of Cinnamomum extract, i.e. the optimum anti-diabetic activity and acts through all possible modes of action.

It has been found, however, that not all mixtures obtained by admixing known plant extracts in an arbitrary ratios display the optimum anti-diabetic qualities required. It has been found that in case of an arbitrary mix of two or more plant extracts, the insulin mimetic activity shown by the mixture might become more pronounced as compared to that shown by the individual extracts, however at the same time, the insulin sensitization activity of the mix might get restricted and vice versa.

A technique as disclosed in the instant invention must therefore be applied to formulate a mixture composition of plant extracts which synergies each species individual anti diabetic effect to produce a multi dimensional, combinatorial anti diabetic effect. Furthermore, it is noted that the various extract must be added in particular determined ratio only. Scientists have derived a combination, which shows the optimum synergistic effect of the desired plant extracts, mixed in optimum quantities. Moreover such mixtures has been selected which show desired effect on desired cell sites and tissues. The mixtures have an optimized quantity of each plant extract mixed to obtain insulin mimetic and sensitization activity at the desired sites.

The heart of the invention therefore lies in the creation of an improved plant extract mixture, wherein the anti diabetic activities of each of the plant extracts is potentiated and synergised by the activity of the other.

In yet another embodiment of the invention, there is provided a process for obtaining plant extracts possessing improved hypoglycemic properties. The processes comprises the steps (a) obtaining plant material from one or more plants (b) obtaining an extract from the plant material by contacting the plant material with an aqueous, an ethanolic or an organic solvent, or a combination thereof, thereby providing one or more plant extracts (c) treating the extract with reagents/decolorizing, deodorizing, debittering agents to obtain an organoleptically improved product (d) analyzing the plant extracts for free radical scavenging potential, intestinal alpha-glucosidase inhibition potential, insulin mimetic activity and insulin sensitizing activity, in-vitro screening of plant extracts for glucose uptake and the in-vivo efficacy studies (e) mixing the plant extracts in predetermined ratios.

### Extraction of the Plant Material by Solvent Extraction Process

The plant material employed in the extraction process can be the entire potential plant, or it can be one or more distinct tissues from the plant for example, leaves, seeds, roots, stems, flowers, or various combinations thereof but preferably the seed of the plant. If desired, the plant material can be treated prior to extraction, for example, by drying, freezing, lyophilizing, or some combination thereof. If desired, the plant material can be fragmented and/or homogenized by some means such that a greater surface area is presented to the solvent. For example, the plant material can be crushed or sliced mechanically, using a grinder or other device to fragment the plant parts into small pieces or particles, or the plant material can be frozen in liquid nitrogen and then crushed or fragmented into smaller pieces.

The solvent used for the extraction process can be aqueous, alcoholic or organic, or a combination thereof. In one embodiment of the present invention, plant material is extracted with an aqueous solvent. Examples of suitable solvents include but are not limited to water, buffers, cell media, dilute acids or bases and the like. In an alternate embodiment of the invention, the plant material is extracted with an alcoholic solvent. Examples of suitable alcoholic solvents include, but are not limited to methanol, ethanol, n-propanol, iso-propanol, 2-butanol, ter-butanol, and combinations thereof.

Various extraction processes are known in the art and can be employed in the methods of the present invention. The extract is generally produced by contacting the solid plant material with a solvent with adequate mixing and for a period of time sufficient to ensure adequate exposure of the solid plant material to the solvent such that inhibitory activity present in the plant material can be taken up by the solvent.

The solvent extraction process may be selected from direct and successive extraction types such as extraction from plant parts in soxhlet apparatus or in flasks at room temperature or at higher temperature with polar and/or non-polar solvent (s). Regardless of the number of extraction processes, each extraction process typically is conducted over a period of time between about 6 hours to 24 hours at room temperature. Shaking the suspension can encourage adequate contact of the solvent with the plant material.

The purified extracts or partially purified extracts are concentrated by solvent removal from the original extract and/or fractionated extract, and/or purified extract. The techniques of solvent removal are known to those skilled in the art and include but are not limited to rotary evaporation, distillation (normal and reduced pressure), centrifugal vacuum evaporation and lyophilisation. The extract referred to herein can be produced by any of the two procedures stated hereunder. The procedures laid down herewith are general procedures alterable with variations known to one skilled in the art. These may not in any way be treated as restrictive to the instant invention.

The solvents used in the process may be aqueous, alcoholic or organic solvents well known in the art and include but are not restricted to heterocyclic aromatic compounds, aliphatic compounds, ketones, cyanides, alcohols, nitriles, esters, ether and mixtures of one or more thereof.

1. Powder the plant material and contact it with the solvent.
2. Heat the solution and continue extraction for approximately 3 hours.
3. Filter and collect the plant extract.
4. Concentrate the extract obtained in step 4 by known procedures such as soxhelation.
5. Dry the extract and cool it.
6. Add hexane to the extract of step 6 and perform solvent partitioning
7. Collect the aqueous layer and treat it with a decolorizing/deodorizing agents.

Optionally, the treated extract obtained is freeze dried. The extract dried is stored in container and the same is taken through several in-vitro cells free and cell based bioassay to validate the extract efficacy.

The above-obtained extract is much lighter in color, when compared with the normal extract.

Modifications of the type, which are required to scale up the procedures and such as are standard in the industry, are included with in the realm and scope of the invention laid down. Similarly, modifications as would be readily apparent to those skilled in the art fall within the scope of the invention.

By making use of the processes stated above with variations known to a person skilled in the art, plant extracts of Eugenia and cinnamon were prepared using varied solvents, solubilizers and concentration procedures. Thereafter the extracts were mixed in a ratio as given hereunder to form the desired mixture.

| | | % age addition to the |
|---|---|---|
| S. No. | Plant extract | Plant Extract Mixture |
| 1 | Eugenia extract | 20-80% |
| 2 | Cinnamon extract | 20-80% |

Various synergistic mixtures were prepared by taking the plant extracts in varying concentrations. The same are stated herewith as examples. It may be noted that these examples are merely illustrative and should not in any way be treated restrictive to the invention.

### EXAMPLES

### Example 1

| | | % age addition |
|---|---|---|
| S. No. | Plant extract | to the mixture |
| 1. | Eugenia ethanol extract | 50 |
| 2. | Cinnamon ethanol extract | 50 |
| 3. | Cinnamon water extract | 0 |
| 4. | Eugenia water extract | 0 |

### Example 2

| | | % age addition |
|---|---|---|
| S. No. | Plant extract | to the mixture |
| 1. | Eugenia ethanol extract | 50 |
| 2. | Cinnamon ethanol extract | 0 |
| 3. | Cinnamon water extract | 50 |
| 4. | Eugenia water extract | 0 |

### Example 3

| | | % age addition |
|---|---|---|
| S. No. | Plant extract | to the mixture |
| 1. | Eugenia ethanol extract | 33 |
| 2. | Cinnamon ethanol extract | 33 |
| 3. | Cinnamon water extract | 33 |
| 4. | Eugenia water extract | 0 |

### Example 4

| | | % age addition |
|---|---|---|
| S. No. | Plant extract | to the mixture |
| 1. | Eugenia ethanol extract | 20 |
| 2. | Cinnamon ethanol extract | 40 |
| 3. | Cinnamon water extract | 40 |
| 4. | Eugenia water extract | 0 |

### Example 5

| | | % age addition |
|---|---|---|
| S. No. | Plant extract | to the mixture |
| 1. | Eugenia ethanol extract | 60 |
| 2. | Cinnamon ethanol extract | 5 |
| 3. | Cinnamon water extract | 20 |
| 4. | Eugenia water extract | 10 |

### Example 6

| | | % age addition |
|---|---|---|
| S. No. | Plant extract | to the mixture |
| 1. | Eugenia ethanol extract | 0 |
| 3. | Cinnamon ethanol extract | 5 |
| 4. | Cinnamon water extract | 75 |
| 4. | Eugenia water extract | 20 |

### Example 7

| | | % age addition |
|---|---|---|
| S. No. | Plant extract | to the mixture |
| 1. | Eugenia ethanol extract | 10 |
| 2. | Cinnamon ethanol extract | 75 |
| 3. | Cinnamon water extract | 5 |
| 4. | Eugenia water extract | 10 |

### Example 8

| | | % age addition |
|---|---|---|
| S. No. | Plant extract | to the mixture |
| 1. | Eugenia ethanol extract | 75 |
| 2. | Cinnamon ethanol extract | 20 |
| 3. | Cinnamon water extract | 5 |
| 4. | Eugenia water extract | 0 |

### Example 9

| | | % age addition |
|---|---|---|
| S. No. | Plant extract | to the mixture |
| 1. | Eugenia ethanol extract | 0 |
| 2. | Cinnamon ethanol extract | 20 |
| 3. | Cinnamon water extract | 5 |
| 4. | Eugenia water extract | 75 |

### Test to Determine the Anti-Diabetic Efficacy of Plant Extract Mixture

### Example 10

### Insulin Mimetic Activity Shown by Plant Extract Mixtures

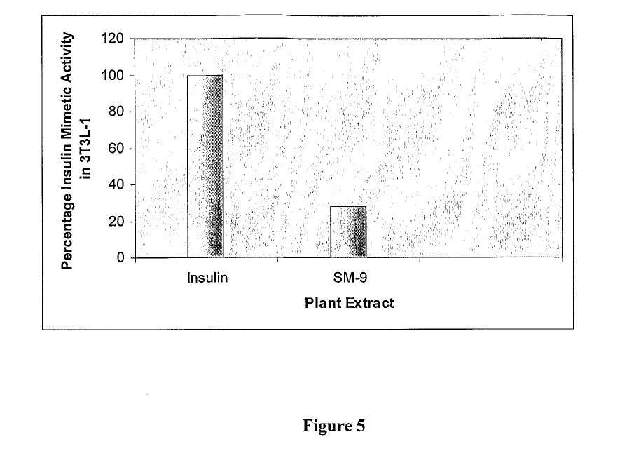

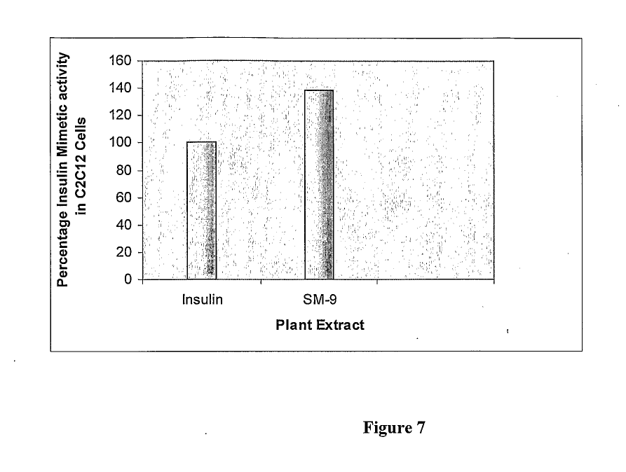

Insulin mimetic activity was tested in both 3T3-1 adipocyte cells and C2C12 myocyte cells. Two different concentration of plant extract were used. The extract shows insulin mimetic activity in varying concentration. The results obtained are shown in FIGS. 5 and 7. These are clearly indicative of the enhanced insulin mimetic activity seen in the plant extract mixtures over and above the individual plant extracts. In FIG. 5 the black bar graph shows the insulin mimetic activity by cinnamomum plant extract individually. In FIG. 7 the black bar graph shows the insulin mimetic activity individually by Eugenia extract. The graph in themselves are self explanatory, indicate though the synergism stated in the invention.

### Example 11

### Insulin Sensitization Activity Shown by Plant Extract Mixtures

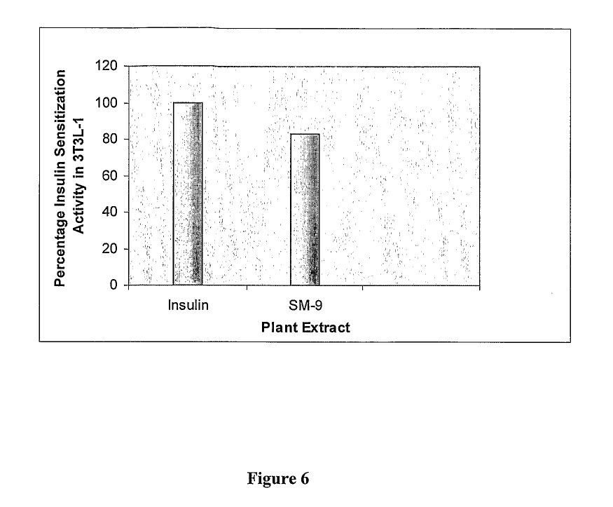

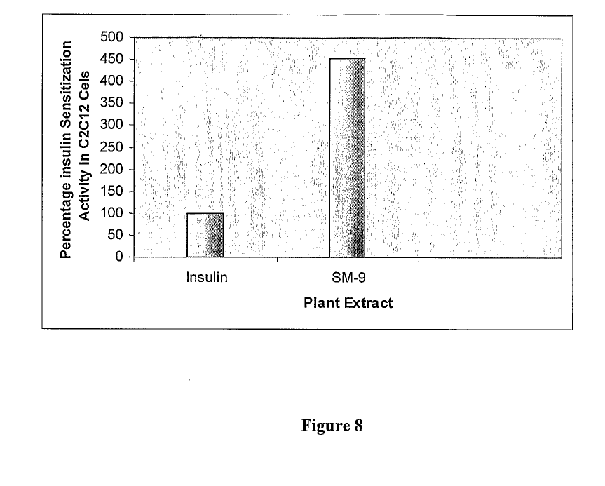

FIGS. 6 and 8 depict the enhanced insulin sensitization activity shown by the plant extract mixtures in comparison to an individual plant extract when seen in adipocytes and myoblasts. In the FIG. 8 the black bar graph shows the insulin sensitization activity by cinnamon individually.

These FIG. 8 Shows a comparative bar graph wherein the insulin sensitization activity of plant extract mixture is compared with the individual plant extract insulin sensitization activities of cinnamon. Herein the black bar shows the insulin sensitization obtained from cinnamon. The graph in itself is indicative of an enhanced insulin sensitization activity produced in all plant extract mixture over and each individual plant extract used to prepare the mixture composition.

### Example 12

### Tests to Determine the Free Radical Scavenging Potential of the Plant Extract Mixture

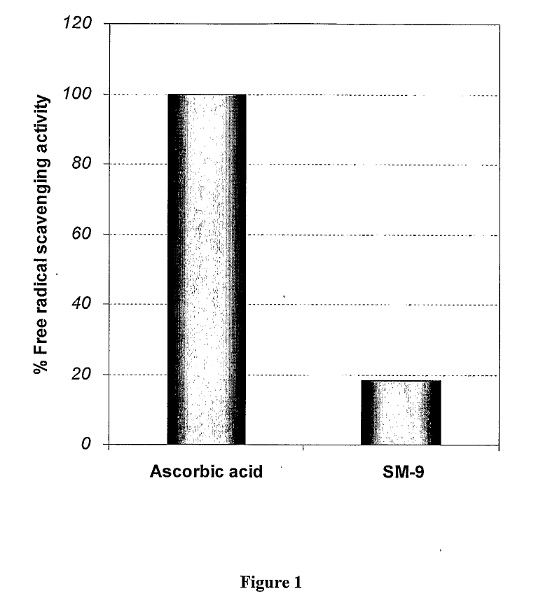

In the present invention the isolated extracts were used to estimate its free radical scavenging potency relative to ascorbic acid by using calorimetric-DPPH method (Polterait O. (1997) Anti Oxidants and free-radical Scavengers of Natural origin Current Org. Chem. 1. 415-440). The SM-9 extract isolated from the plant parts showed nearly about 18% of free radical scavenging potency equivalent to that of ascorbic acid. The result are represented in the form of bar graph in FIG. 1.

### Example 13

### Estimation of Total Polyphenols and its Hypoglycemic Effects of Plant Extracts

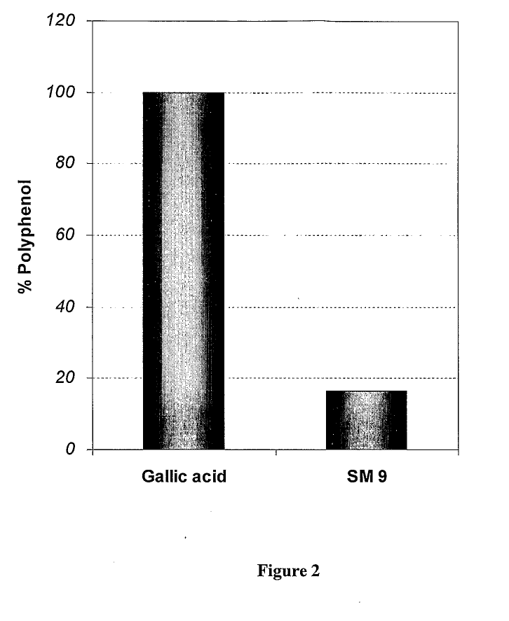

In the current invention, the total polyphenol content of the SM-9 extract was estimated relative to gallic acid using calorimetric-Singleton method (Singleton, V. L. and Ropssi, J. A. Jr (1965). Calorimetry of total phenolics with phosphomolybdic-phosphotungstic acid reagent, Am. J. Enol. Vitig. 16: 144-158). The SM-9 extract showed about 18% total phenol content equivalent to gallic acid clearly indicative of the potential beneficial effects the extracts possess with respect to the management of diabetes and its medicative properties. The results are represented in the form of bar graph in FIG. 2.

### Example 14

### Intestinal Alpha-Glucosidase Inhibition Potential

The inhibition of degradation of oligosacharrides (carbohydrates having 2 to 10 glucose residues connected by 1-4 or 1-6 alpha-D-glycosidic linkage) into monosaccharides by alpha-glucohydrolase-catalyzed enzymatic reactions is tested for SM-9 extract using Calorimetric—para-nitro-phenyl (pNP) release method using pNP-a-D-glucoside (Halvorson. H, ‘Methods in enzymology’ Vol 8, Academics Press, New York, 1966, p 559-562). The plant extract mixture shows greater α-glucosidase inhibition potential (IC50 value of 147.06 μg/ml) relative to the commercially available—α glucosidase inhibitor, acarbose (IC50 value of 146.55 μg/ml) for 0.2 α-glucosidase enzyme units at standard enzymatic reaction conditions. The results are depicted in FIG. 9. In this figure the alpha glucosidase activity is shown by different extracts. It is evident that both individually and when formulated as a mixture used individually its alpha glucosidase activity is far less compared to the plant extract mixtures.

### Example 15

### In-Vivo Efficacy Screening Using Sprague Dawley Rats

In-vivo efficacy screening of SM-9 extract was done using Sprague dawley rats and the procedure undertaken is as follows:

Sprague dawley rats weighing ˜250 g with a variation of ±20% of the mean weight are selected for In-vivo efficacy screening of SM-9 extract. The number of animals per dose group is five and is kept for examination and acclimatization for a week period at the start of the study. The total number of dose groups is six along with vehicle control. The different groups are G1-Vehicle control, G2-Diabetic control, G-3 Diabetic animals treated with Pioglitazone (Standard anti-diabetic drug). G-4 SM-9 extract 50 mg/kg-body weight, G-5 SM-9 extract 125 mg/kg-body weight, G-6 SM-9 extract 200 mg/kg-body weight. Blood is collection under CO2 anesthesia by retro orbital (ROP) plexus method and FBG analysis. Rats are fed with 5% glucose water for two days before STZ injection and are fasted for 12 hours before administering streptozotocin. The rats are injected intra-peritoneal with 45 mg/kg-body weight of streptozotocin in a citrate buffer ph: 4.5 and allowed for week recovery. Rats are fasted for 12 hours after 7th day of STZ injection and blood collected in heparin as the anti-coagulant, under CO2 anesthesia and analyzed for FBG. Rats that have the range of FBG beyond 250 mg/dl and above are considered for the study. On 8th day onwards, the in-vivo efficacy screening of SM-9 at different concentrations is started and the dosing is 50, 125 and 200 mg per kg body with 0.5 percent of carboxyl-methyl-cellulose (CMC) as vehicle.

### Example 16

### Screening of SM-9 Extract at 50, 125 and 200 mg/kg Body Weight

Selection, randomization and grouping of the animals into different treatment groups with the FBG range of 250 mg/dl is undertaken as above. The dosing formulations are prepared freshly each day 0.5% CMC was used as the vehicle. The test article SM-9 is administered by oral gavage to each rat daily, for 35 consecutive days. The animals were dosed at approximately the same time each day where possible using a stainless steel intubation needle fitted onto a suitably graduated glass syringe. The dosage volume administered to individual rat was adjusted according to its most recently recorded body weights. Treatment in this manner continued once a day, seven days a week, for a total period of 35 days. Vehicle control group animals are treated with the vehicle only at the same dosage volume i.e. 10 ml/kg body weight. The Groups include: G1-Vehicle control, G2-Diabetic control, G-3 Diabetic animals treated with Pioglitazone (Standard anti-diabetic drug) 20 mg/kg-body weight. G-4 SM-9 extract 50 mg/kg-body weight, G-5 SM-9 extract 125 mg/kg-body weight, G-6 SM-9 extract 200 mg/kg-body weight. Throughout the study, all cages were checked early on each working day and again in the afternoon and evening to look for dead or moribund animals to allow necropsy examination to be carried out during the working hours of that day. All rats that will be killed in extremes, or found dead in the cage were subjected to detailed necropsy examination. All signs of ill health, together with any behavioral changes or reaction to treatment are recorded for individual animals. Dated and signed records of appearance, change and disappearance of clinical signs are maintained in clinical history sheets for individual animals. The parameters that are observed or included in the study were postprandial glucose, fasting blood glucose (FBG), body weight and feed consumption. Blood collection by ROP on week 1, 2 and 5 for the above said parameters. The animals selected for the study are treated with the extract at 3 different dose levels for 35 days. Necropsy Examination: On completion of 35 days of treatment period, all surviving rats will be sacrificed by exsanguinations under CO₂ anesthesia. Complete necropsies were carried out on all animals including those, which died during the study.

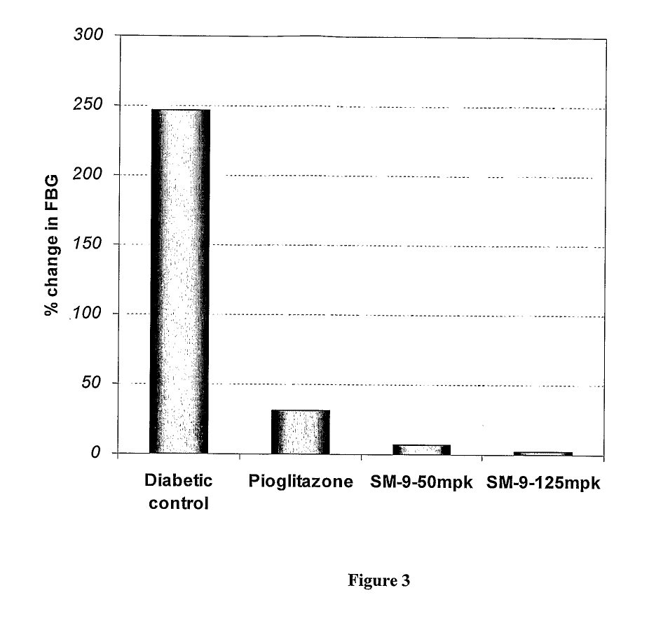

The postprandial blood glucose lowering potential of SM-9 extract at different concentrations are illustrated in FIG. 3. The diabetic control rat group showed 240% increase in their postprandial blood glucose levels compared to the baseline values of the same group at the start of the study. While the groups treated with pioglitazone, SM-9 extract at 9-125 mpk (milligram per kg body weight) did not show any significant percent change from their baseline values.

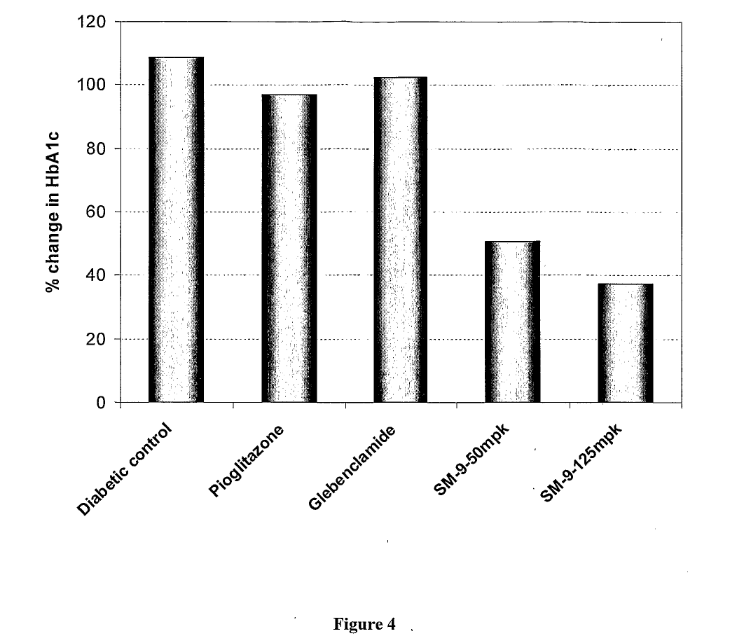

The fasting blood glucose lowering potential of the SM-9 extract at different concentrations are illustrated in FIG. 4. The diabetic control rat group showed 150% increase in their fasting blood glucose levels compared to the baseline values of the same group at the start of the study. The group treated with pioglitazone showed about 98.% increase, while glebenclamide showed about 110%, SM-9 extract at 50, 125 mpk showed about 50% and 39.36% increase respectively from their baseline values at the end of 35 days. The glucose lowering response of 9-125 mpk SM-9 mixture. The results have been represented in FIG. 4.

### Example 17

### Use of the Extract as a Therapeutic Composition

The present invention envisages the method of treating diabetes and other related diseases thereof by administering an effective amount of the therapeutic composition comprising the mixture plant extract or the screened plant extracts purified there from in combination. The therapeutic compositions of the invention can be administered alone or in combination with one or more standard anti-diabetic therapeutics. The present invention also contemplates the administration of sub-optimal doses of the therapeutic composition, for example, chemotherapeutic drug(s), in combination with the therapeutic composition.

Thus, in one embodiment of the present invention, in order to prepare a therapeutic combination, one or more plant extracts is first selected and then the efficacy of the extract(s) in controlling diabetes and maintaining glucose homeostasis is determined using standard techniques as one of those outlined above. The efficacy of the one or more plant extract alone is then compared to the efficacy of the one or more plant extract in combination with varying amounts of another component i.e., another plant extract. The invention also contemplates the combination of the plant extract mixture with another synthetic drug inhibitor. A combination that demonstrates therapeutic index in comparison to the individual properties is considered to be an effective combination.

The formulations of the present invention contain at least an effective amount of the therapeutic composition. The effective amount is considered to be that amount of the composition, in weight percent of the overall formulation, which must be present in order to produce the desired therapeutic effect. As would be apparent to one skilled in art, the effective amount may vary, depending upon, for example the disease to be treated and the form of administration. In general the therapeutic composition will be present in an amount ranging from about 1% to 100% by weight of the formulation, 10% to about 90% by weight of the formulation, 20% to about 80% by weight of the formulation, 30% to 70% by weight of the formulation, from about 40% to 60% by weight of the formulation and about 50% by weight of the formulation.

The present invention contemplates the use of the therapeutic compositions at various stages in the disease development and progression, including in the treatment of early stage, or advanced and/or aggressive stage of hyperglycemia, diabetes or related disorders. The administration of the therapeutic composition comprising the isolated and screened extracts to mammal having an early stage of the disorder can help to attenuate the progression of the disease.

The dosage of the therapeutic composition to be administered is not subject to defined limits, but will usually be an effective amount. However it will be understood that the actual amount of the composition to be administered will be determined by a physician, in the light of the relevant circumstances, including the exact condition to be treated, the chosen route of administration, the actual composition administered, the age, the weight, and the response of the individual patient and the severity of the patient's symptoms. The dosage ranges are not intended to limit the scope of the invention in any way.

The therapeutic compositions comprising the mixture of plant extract are not limited to only those for humans but also include those for various animals, in particular, other mammals. Therefore, the food compositions include foods for animals such as cats, dogs, and the like pets, and the medical compositions include those for animals other than humans.

### Example 18

### Modes of Administration

For administration to a mammal, the therapeutic composition can be formulated as a herbal or naturopathic formulation such as phytoceuticals or nutraceuticals, for oral, topical, rectal or parenteral administration or for administration by inhalation or spray. The phytoceutical or naturopathic formulation may comprise the one or more plant extracts in dosage unit formulations containing the conventional non-toxic physiologically acceptable carriers, adjuvant and vehicles.

The herbal or naturopathic formulations may be in a form suitable for oral use, for example, as tablets, troches, lozenges, dispersible powders or granules, emulsion hard or soft capsules, or syrups or elixirs. The therapeutic compositions of the invention may be formulated as phytoceuticals, or nutraceuticals. Phytoceuticals may optionally comprise other plant-derived components and can therefore be delivered by such non-limiting vehicles as teas, tonics, juices or syrups. Nutraceuticals contemplated by the present invention may provide nutritional and/or supplemental benefits and therefore be delivered, for example as foods, dietary supplements, extracts, beverages or the like. Phytoceutical and nutraceuticals can be administered in accordance with conventional treatment programs and/or may be a part of the dietary or supplemental program. Formulations intended for oral use may be prepared according to methods known in art for the manufacture of herbal compositions and may contain one or more agents selected from the group of flavoring agents, coloring agents and preserving agents in order to provide palatable preparations.

Tablets contain the active ingredient in admixture with suitable non-toxic physiologically acceptable excipients including, for example, inert diluents, such as calcium carbonate, lactose, calcium phosphate or sodium phosphate; granulating and disintegrating agents, such as corn starch, or alginic acid, binding agents, such as starch, gelatine or acacia, and lubricating agents, such as magnesium stearate, stearic acid or talc. The tablets can be uncoated, or they may be coated by known techniques in order to delay disintegration and absorption in the gastrointestinal tract and thereby provide a sustained action over a longer period.

Various additives or carriers can be incorporated into the orally delivered pharmaceutical naturopathic formulations or the invention. Optional additives of the present composition include, without limitation, phospholipids, such as phosphatidyl glycerol, phosphotidyl inositol, phosphotidyl serine, phosphotidyl choline, phosphotidyl ethanolamine as well as phosphatidic acids, ceramide, cerebrosides, sphingomyelins and cardiolipins.

Herbal or naturopathic formulations for oral use may also be presented as hard gelatin capsules wherein the active ingredient is mixed with an inert solid diluent, for example, calcium carbonate, calcium phosphate or kaolin, or as soft gelatine capsules wherein the active ingredient is mixed with water or an oil based medium such as peanut oil, liquid paraffin or olive oil.

Oily suspensions may be formulated by suspending the plant extract(s) in a vegetable oil, for example, arachis oil, olive oil, sesame oil or coconut oil, or in a mineral oil such as liquid paraffin. The oily suspensions may contain a thickening agent, for example beeswax, hard paraffin or acetyl alcohol. Flavoring agents may be added to provide palatable oral preparations. These formulations can be preserved by the addition of an anti-oxidant such as ascorbic acid.

Dispersible powders and granules suitable for preparation suitable for an aqueous suspension by the addition of water provide the active ingredient in admixture with dispersing or wetting agent, suspending agent and one or more preservatives. Suitable dispersing or wetting agents and suspending agents, sweetening, flavoring and coloring agents may also be present.

In a further aspect of the invention there is provided a comestible, that is to say, a foodstuff comprising at least an extract mixture of the invention, typically in dried form, such as in a lyophilised form. The skilled addressee will appreciate that such cosmetibles may contain more than one extract of the invention and may be used. Such foodstuffs may be used in a prophylactic manner and may contain further extracts having a similar function to the first added extract or further added extracts may be added that have a different prophylactic function. Thus a foodstuff could either comprise extracts that provide for a comestible having a single functional aspect, or a comestible may have a multi-functional prophylactic effect against two or more disease types. It is thought that a multi-functional role could be assigned to pharmaceutical formulations comprising two or more extracts possessing dissimilar therapeutic or prophylactic properties designed either for prophylaxis or for the treatment of more than one disease(s) in a mammal, particularly in a human.

The type of foodstuff or comestible to which at least an extract of the invention may be added includes any processed food such as confectionaries, baked products including breads such as loafs, and flat breads such as pitta bread, naan bread and the like, cakes, snack foods such as museli bars, compressed dried fruit bars, biscuits, dairy products such as yoghurts, milk and milk-based products such as custards, cream, cheese, butter and crème fraiche, simulated dairy food product such as Elmlea products, fruits and vegetable juices, aerated drinks, such as carbonated soft drinks and non-aerated drinks such as squashes, soya milk, rice milk and coconut milk and the like, pastas, noodles, vegetables, seed and nut oils, fruited oils such as sunflower oil, rapeseed oil, olive oil, walnut, hazelnut, and sesame seed oil and the like, and frozen confectionaries such as ice cream, iced yoghurts and the like.

The present water and ethanol based plant extracts can also be used for the management of diabetic related disorders, which are general or local diseases directly or local diseases directly or indirectly caused by diabetes, specific examples thereof are diabetic acidosis, diabetic xanthoma, diabetic myatrophy, diabetic ketosis, diabetic coma, diabetic stomach disorders, diabetic gangrene, diabetic ulcer, diabetic diarrhea, diabetic microangiopathy, diabetic ulerosclerosis, diabetic cardiomyopathy, diabetic neuropathy, diabetic blister, diabetic cataract, diabetic dermatitis, diabetic scleredema, diabetic retinopathy, diabetic necrobisis, lipoidica, diabetic blood flow obstractions etc.

The present invention contemplates the large-scale preparation of the selected mixture of the plant extract invention. Such extracts can be prepared on a commercial scale by repeating the extraction process that lead to the isolation of the extract of interest. The small-scale extraction procedure can simply be scaled up and additional steps of quality control can be included to ensure reproducible results for the resulting extracts.

Also contemplated by the present invention are the modifications to the small-scale procedure that may be required during the scale up for the industrial level production of the extract. Such modifications may include for example, alterations to the solvent being used or to the extraction procedure per se employed in order to compensate for variations that occur during the scale-up and render the overall procedure more amenable to industrial scale production, or more cost effective. Modifications of this type are standard in the industry and would be readily apparent to those skilled in the art.

While the invention has been described in connection with specific and preferred embodiments, thereof it is capable of further modification without departing from the spirit and scope of the invention. This application is intended to cover all variations uses, or adaptations of the invention, following in general, the principles of the inventions and including such departures from the present disclosure as come with known or customary practice within the art to which the invention pertains, or as are obvious to person skilled in the art, at the time the departure is made. It should be appreciated that the scope of this invention is not limited to the detailed description of the invention herein above, which is intended merely to be illustrative, but rather comprehends the subject matter defined by the claims. Although the present invention has been described in considerable detail with reference to certain preferred version thereof, other version is possible. Therefore, the spirit and scope of the appended claims should not be limited to the description of the preferred version contained herein.

### Example 19

### Non-Toxicity Data

Acute oral toxicity study of Antidiabetic extract—SM 9 in Sprague Dawley rats was performed in compliance with the OECD Guidelines for testing of chemicals, Section 4, No. 423—Acute Oral Toxicity—Acute Toxic class Method, Adopted 17 Dec., 2001. The method uses Pre-defined doses and the results allow substance to be ranked and classified according to globally Harmonised system (GHS) for classification of chemicals which causes acute toxicity.

In the present study, single oral administration of Antidiabetic Extract—SM 9, Suspended in corn oil, was made to groups of three female Sprague Dawley rats in step-wise manner to assess its acute toxicity. Following the starting dose of 2000 mg/kg, which was also repeated in the second step of the test, Antidiabetic extract—SM 9 did not cause death of any of treated females. Also, no abnormal clinical signs were observed in six female rats.

In the next step of the study Antidiabetic Extract—SM 9 was further tested at the dose of 5000 mg/kg, on three female rats in stepwise manner. The test article did not cause death of any of the female rats treated at 5000 mg/kg after dosing and also did not induce any signs of evident toxicity.

Antidiabetic Extract—SM 9, at the dose level of 200 mg/kg and 5000 mg/kg body weight, did not adversely affect body weight gain by treated rats during the 14 days observation period, post-treatment .the test article did not induce any gross pathological alteration in the tissues/organs of the treated rats as was evident during the terminal necropsy.

Based on these results, and according to the globally Harmonised System (GHS) for Classification of chemicals which cause acute toxicity, OECD series on testing and assessment, Number 33; harmonized integrated classification system for human health and environmental hazards of chemical substances and Mixtures [ENV/JM/MONO (2001) 6]”, the test article antidiabetic extract—SM 9 has to be classified as GHS Category 5 or Unclassified for the obligatory labeling requirement for oral toxicity. This category indicates that following acute oral exposure to antidiabetic Extract—SM 9 in female rats, the LD50 value is expected to expected to exceed 5000 mg/kg.

1. A synergistic herbal composition for diabetes and related disorders comprising extracts of plant Eugenia of concentration ranging between 10-80% and plant Cinnamomum of concentration ranging between 10-80% optionally along with a pharmaceutically acceptable excipient.

2. The synergistic herbal composition of claim 1, wherein the extract is obtained from plant parts selected from the group consisting of root, shoot, leaf, seed, and fruit and the whole plant.

3. The synergistic herbal composition of claim 2, wherein the extract is obtained from seeds of plants.

4. The synergistic herbal composition of claim 1, wherein the extract is either an aqueous or an organic extract.

5. (canceled)

6. The synergistic herbal composition of claim 4, wherein the organic extract is selected from the group consisting of an alcoholic extract and an ethanolic extract.

7. (canceled)

8. The synergistic herbal composition of claim 1, wherein the excipient is selected from the group consisting of additives, gums, sweeteners, coatings, binders, disintegrants, lubricants, disintegration agents, suspending agents, granulating agents, solvents, colorants, glidants, anti-adherents, anti-static agents, surfactants, plasticizers, emulsifying agents, flavoring agents, viscocity enhancers, antioxidants and mixtures thereof.

9. The synergistic herbal composition of claim 1, wherein the composition is formulated into dosage forms selected from the group consisting of liquid, troches, lozenges, powder, granule, capsule, tablet, patch, gel, emulsion, cream, lotion, dentrifice, spray, drop, suspension, syrups, elixirs, phyotceuticals and neutraceuticals, and wherein the composition does not induce side effects.

10. (canceled)

11. A method of treating diabetes and related disorders in a subject in need thereof by administering the composition comprising extracts of plant Eugenia of concentration ranging between 10-80% and plant Cinnamomum of concentration ranging between 10-80% optionally along with pharmaceutically acceptable excipients.

12. The method of claim 11, wherein the related disorders comprise diabetic acidosis, diabetic xanthoma, diabetic myatrophy, diabetic ketosis, diabetic coma, diabetic stomach disorders, diabetic gangrene, diabetic ulcer, diabetic diarrhea, diabetic microangiopathy, diabetic uterosclerosis, diabetic cardiomyopathy, diabetic neuropathy, diabetic nephropathy, diabetic blister, diabetic cataract, diabetic dermatitis, diabetic scleredema, diabetic retinopathy, diabetic necrobiosis lipoidica, diabetic and blood flow obstructions, and wherein the subject is selected from the group consisting of an animal and a human being, and wherein the composition does not induce side effects.

13. (canceled)

14. The method of claim 11, wherein the excipients are selected from the group consisting of additives, gums, sweeteners, coatings, binders, disintegrants, lubricants, disintegration agents, suspending agents, granulating agents, solvents, colorants, glidants, anti-adherents, anti-static agents, surfactants, plasticizers, emulsifying agents, flavoring agents, viscocity enhancers, antioxidants and mixtures thereof.

15. The method of claim 11, wherein the composition is formulated into dosage forms selected from the group consisting of liquid, troches, lozenges, powder, granule, capsule, tablet, patch, gel, emulsion, cream, lotion, dentrifice, drop, suspension, syrups, elixirs, phyotceuticals and neutraceuticals.

16. (canceled)

17. A process for preparation of a synergistic herbal composition for diabetes and related disorders, wherein the process comprises the steps of:
a) Obtaining extracts of Eugenia and Cinnamomum separately using aqueous or organic solvents; and
b) Formulating the extracts to arrive at the synergistic composition.

18. The process of claim 17, wherein the extract is obtained from plant parts selected from the group consisting of root, shoot, leaf, seed, and fruit and the whole plant.

19. The process of claim 18, wherein the extract is obtained from seeds of the plants.

20. The process of claim 10, wherein the organic solvents are selected from the group consisting of heterocyclic aromatic compounds, aliphatic compounds, ketones, cyanides, alcohols, nitriles, esters, ether and mixtures thereof.

21. The process of claim 20, wherein the organic solvent is ethanol.

22. The process of claim 17, wherein the extract obtained is treated with organoleptic agents selected from the group consisting of decolorizing agents, deodorizing agents, debitterizing agents and mixtures thereof, and wherein the decolorizing agents are selected from the group consisting of peroxides, carbon, metals, organic solvents, and mixtures thereof.

23. (canceled)

24. The process of claim 22, wherein the decolorizing agent is hydrogen peroxide.

25. The process of claim 22, wherein the deodorizing agents are selected from the group consisting of peroxides, carbon, organic solvents and mixtures thereof.

26. The process of claim 22, wherein the deodorizing agent is preferably ketone.

27. The process of claim 17, wherein the composition is formulated into a dosage form selected from the group consisting of liquid, troches, lozenges, powder, granule, capsule, tablet, patch, gel, emulsion, cream, lotion, dentrifice, drop, suspension, syrups, elixirs, phyotceuticals and neutraceuticals.
